(19) United States
(12) Patent Application Publication
(10) Pub. No.: US 2026/0357032 A1
(43) Pub. Date: Nov. 12, 2026

(54) LOW TEMPERATURE BASE CATALYZED METHANOLYSIS OF POLYESTERS

(71) Applicants: Alliance for Sustainable Energy, LLC, Golden, CO (US); AMAZON.COM SERVICES, LLC, Seattle, WA (US)

(72) Inventors: Julia Bryant CURLEY, Denver, CO (US); Katrina Marie KNAUER, Denver, CO (US); Gregg Tyler BECKHAM, Golden, CO (US); David Gregory BRANDNER, Golden, CO (US); Jason Scott DESVEAUX, Westminster, CO (US); Robert David ALLEN, Golden, CO (US); Alan Jon JACOBSEN, Issaquah, WA (US)

(21) Appl. No.: 19/157,573

(22) PCT Filed: Feb. 16, 2024
(86) PCT No.: PCT/US24/16091

§ 371 Date: Aug. 18, 2025

Related U.S. Application Data
(60) Provisional application No. 63/485,294, filed on Feb. 16, 2023.
(60) Provisional application No. 63/514,732, filed on Jul. 20, 2023.

(51) Int. Cl. C08J 11/28; C08K 5/17

(52) U.S. Cl. CPC C08J 11/28; C08K 5/17; C08J 2367/00

(57) ABSTRACT

Disclosed herein are methods and compositions for developing an energy-efficient chemical processing technology that can break down, or deconstruct, plastics into valuable feedstocks that can be used to make the original plastics that were fed into this process (closed-loop recycling) or new plastics altogether (open-loop recycling) and develop new plastics that could be made from the chemicals coming out of the deconstruction process.

### CROSS-REFERENCE TO RELATED APPLICATIONS

This application claims priority under 35 U.S.C. § 119 to U.S. provisional patent application No. 63/485,294 filed on 16 Feb. 2023 and U.S. provisional patent application No. 63/514,732 filed on 20 Jul. 2023, the contents of which are hereby incorporated in their entirety. This application is a national stage 371 application of PCT/US2024/016,091 filed on 16 Feb. 2024, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

New materials and processes for making and deconstructing plastic packaging materials that are net-zero carbon, fully circular, and harmless to the environment is a benefit to the earth and society. There is a need for energy-efficient chemical recycling processes for packaging materials, and to synthesize new or existing plastics from the plastic deconstruction products. There is also a need for designing plastics with a wide range of material properties for packaging applications while also containing chemical linkages known to deconstruct by current processes that would result in recyclability in a materials agnostic process which will minimize the need for costly material separation, and which can tolerate a range of expected contaminates.

### SUMMARY

In an aspect, disclosed herein are methods for making at least one scalable chemical processing technology that can depolymerize and separate building blocks from current and emerging streams of plastics with ester linkages.

In an aspect, disclosed herein are methods for catalysis-enabled approaches for conversion of intermediates into either the same recyclable plastic (closed-loop) or a polyethylene (PE)-like replacement (open-loop).

In an aspect, disclosed herein is a method for converting a polymer containing an ester-bond into monomers comprising the step of contacting the polymer with an amine in a solution comprising alcohol at a temperature of from 100 to 190 degrees Celsius. In an embodiment, the alcohol is methanol. In an embodiment, the amine is a tertiary amine. In an embodiment, the amine is selected from the group consisting of DMEA or TEA. In an embodiment, the boiling point of the amine differs from the boiling point of the alcohol by at least 24 degrees Celsius. In an embodiment, the polymer is selected from the group consisting of PET, PVL, PBT, PET, PLA, PBAT, PHAs, PCL and PBS. In an embodiment, the monomers are selected from the group consisting of DMT, ML, DMA, DMS, EG and BDO. In an embodiment, the molar ratio of alcohol to the polymer is 8 to 1. In an embodiment, the weight percent of the amine is about 5 weight percent of the weight of the reaction components. In an embodiment, polymer comprises 20 to 60 weight percent of the reaction components. In an embodiment, about 55 mole percent of the polymer is converted into monomers after a reaction time of about 30 minutes. In an embodiment, about 70 mole percent of the polymer is converted into monomers after a reaction time of about 2 hours.

In an aspect, disclosed herein is a method for converting a mixture of different ester-bond containing polymers into monomers comprising the step of contacting the mixture of polymers with an amine in a solution comprising alcohol at a temperature of from 100 to 190 degrees Celsius. In an embodiment, the alcohol is methanol. In an embodiment, the amine is a tertiary amine. In an embodiment, the amine is selected from the group consisting of DMEA or TEA. In an embodiment, the boiling point of the amine differs from the boiling point of the alcohol by at least 24 degrees Celsius. In an embodiment, the mixture of polymers contains polymers selected from the group consisting of PET, PVL, PBT, PET, PLA, PBAT, PHAs, PCL and PBS. In an embodiment, the monomers are selected from the group consisting of DMT, ML, DMA, DMS, EG and BDO. In an embodiment, the weight percent of the amine is about 5 weight percent of the weight of the reaction components. In an embodiment, the mixture of polymers comprises 20 to 60 weight percent of the reaction components. In an embodiment, about 80 mole percent of the polymer mixture is converted into monomers. In an embodiment, greater than about 80 mole percent of the polymer mixture is converted into monomers after a reaction time of about 3 hours.

In an aspect, disclosed herein are compositions comprising a polymer resulting from the polymerization of any of the monomers resulting from the use of any of the methods disclosed herein.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts a scheme for flexible closed-loop or open-loop deconstruction processes of materials with ester linkages.

FIG. 2 depicts dimethyl terephthalate yield over time from the deconstruction of PET into using the reaction conditions depicted in Scheme 1 (DMEA or TEA at 5 weight percent; 8:1 molar ratio of MeOH to PET at 170 degrees Celsius).

FIG. 3 depicts the mole percent conversion of PLA and PBAT to products using reaction conditions depicted in Scheme 2 (DMEA 5 weight percent; 8:1 molar ratio of MeOH to either PLA or PBAT at 120 degrees Celsius, reacting for 30 or 60 minutes). As depicted in FIG. 3, using the reaction conditions of Scheme 2, PLA undergoes about a 55 mole percent conversion to products reacting for 30 minutes under the conditions of Scheme 2 and about a 75 mole percent conversion to products reacting for 60 minutes under the conditions of Scheme 2. Under the conditions of Scheme 2, PBAT undergoes almost no conversion to products.

FIG. 4 depicts conversion of PET or PLA or PBAT or PBS via reaction conditions depicted in Scheme 3 (DMEA 5 weight percent; 20 weight percent polymer in MeOH at 190 degrees Celsius, reacting for 2 hours). As depicted in FIG. 4, PET is converted to DMT at about an 82 mole percent monomer yield; PLA is converted to ML at about a 90 mole percent yield; PBAT is converted to DMT and DMA at about a 70 mole percent yield and an 80 mole percent yield, respectively; and PBS is converted to DMS at about an 80 mole percent yield.

FIG. 5 depicts the conversion of a mixture of PET:PLA:PBAT:PBS at relative molar ratios of 70:14:14:2, to DMT, ML, DMA, DMS, EG and BDO monomers using the reaction conditions of Scheme 4.

### DETAILED DESCRIPTION

Methods disclosed herein improve upon existing methanolysis systems by enabling lower reaction temperatures and pressures, higher polymer loadings, and facile catalyst recovery via distillation while still providing high product yields. Methods disclosed herein can be used to address the growing need for simple and efficient mixed plastics recycling.

Methods disclosed herein use simple, commercially available amines to catalyze the methanolysis of polyesters including PET (polyethylene terephthalate), PLA (polylactic acid), PBAT (polybutylene adipate terephthalate), PBS (polybutylene succinate), PHAs (polyhydroxyalkanoates), PVL (poly(valerolactone)), PBT (poly(butylene terephthalate)), polycarbonates, polyurethanes, and mixtures thereof. These reactions run at high polymer concentrations (20-60 wt %) under mild conditions: no added pressure required, reaction times of less than 6 hours, and temperatures between 100-190° C. Under these conditions, polyesters are deconstructed into methyl ester and diol monomeric products. The products of these depolymerization reactions can be repolymerized to form new polymeric materials or sold as platform chemicals.

Methods disclosed herein can be used on multiple polyesters, including mixed feedstocks. The polymer concentrations used are quite high, increasing efficiency, but the required catalyst loadings are low. The process is scalable and runs at reduced energy and solvent loadings compared to most existing methanolysis systems.

It is contemplated that the methods disclosed herein can use multiple different kinds of alcohols and mixtures of alcohols.

Disclosed herein are methods to create plastics that are net-zero carbon, fully circular, and harmless to the environment. In an embodiment, disclosed herein are plastics that are derived from biobased or waste feedstocks, can be efficiently recycled without diminishing the quality, and can safely breakdown in the soil or ocean if they happen to leak into environment. In an embodiment, disclosed herein are plastics that can be designed at the molecular level to have a range of physical properties and can be recycled in a scalable, low-energy process that minimizes the need for costly material separation and can handle a range of expected contaminates.

In an embodiment, disclosed herein are plastics with ester bonds (also referred to as ester linkages). Most commodity plastics, including polyethylene and polypropylene, are comprised of molecular chains held together by strong carbon-carbon bonds, which makes these materials persistent in the environment and energy-intensive to breakdown using chemical recycling technologies. However, plastics comprised of molecular chains held together by ester linkages, can be more efficiently deconstructed into feedstock materials through known chemical recycling techniques (hydrolysis, enzymatic deconstruction, etc.), and because ester linkages are prevalent in naturally occurring materials, they offer a pathway for the materials to safely breakdown in the environment.

Existing and emerging plastics with ester linkages include polyethylene terephthalate (PET), polylactic acid (PLA), polybutylene adipate terephthalate (PBAT), polycaprolactone (PCL), and polyhydroxyalkanoates (PHAs). The mechanisms used to break an ester linkage can be the same for all these materials, albeit under different conditions. Ultimately, the ease at which ester linkages in these plastics can be broken, either through a chemical process or degradation in a natural environment, depends on the molecular structure and morphology of each specific material.

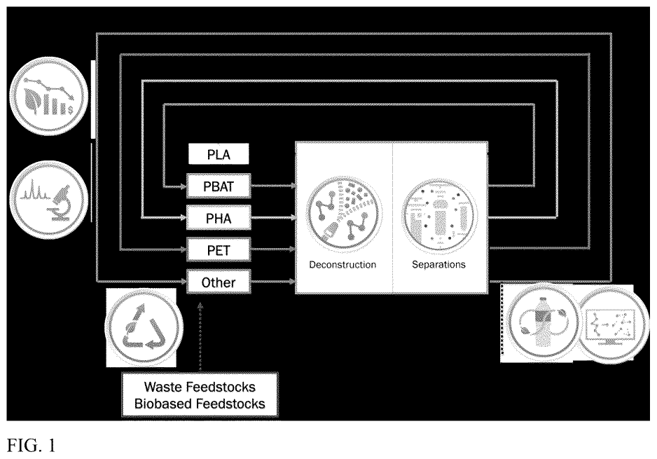

In an embodiment, disclosed herein is an energy-efficient chemical processing technology that can break down, or deconstruct plastics with ester linkages into valuable feedstock that can be used to make the original plastics that were fed into this process (closed-loop recycling) or new plastics altogether (open-loop recycling). By enabling the deconstruction of a range of different plastics using a single processing technology, the need for excessive sortation of the materials before deconstruction is eliminated. This will also accelerate scaling of the technology because of greater available material volumes, and the technology itself will not be dependent on the commercial success of one single material. An embodiment of this new flexible deconstruction process is depicted in a schematic represented as a flywheel where the materials that require the least amount of time and energy to breakdown and recover will lead to lower cost feedstock for new materials production (FIG. 1). As costs come down for a specific material, the use of that material will increase, further reducing costs.

### Feedstock-Agnostic Deconstruction, Separations, and Remanufacturing Processes for Polyesters

In an embodiment, disclosed herein are methods to develop a fully integrated process and corresponding economic, energy, and greenhouse gas (GHG) emissions analyses for a chemical process that is able to deconstruct mixed plastics with ester linkages and enable both closed-loop recycling and open-loop upcycling to film materials.

In another embodiment, disclosed herein are separations approaches to recover pure monomers from mixed intermediates using advanced separations techniques, including process modeling.

In an embodiment, disclosed herein are methods for using catalysis-enabled options to remanufacture RBD plastics with ester-linkages.

In an embodiment, disclosed herein are methods for using enzyme-based processes for deconstruction of mixed plastics with ester-linkages.

In an embodiment, disclosed herein are methods to develop new plastics that can be made from the chemicals coming out of the deconstruction process. In some cases, closed-loop recycling may make the most sense. However, existing plastics with ester linkages do not fully achieve the long-term vision of being net-zero carbon, fully circular, and harmless to the environment. In addition, these available materials have limitations on the achievable physical properties, which in turn, limits their application space. In particular, it is challenging to achieve polyethylene (PE)-like properties with these plastics, which is important for film-based packaging applications.

In an embodiment, disclosed herein are methods useful for synthesizing new plastics with ester linkages.

Methods disclosed herein include the development of film processing for RBD polymers; the characterization of film properties (tensile, tear, slow rate puncture, etc.); and accelerated degradation testing and evaluation of resultant products.

In an embodiment, methods and compositions of matter are disclosed herein that utilize additional feedstock materials, with a focus on waste-based feedstocks and on direct oxidation and tandem hydrogenolysis and oxidation of PE to building blocks for ester-based, RBD PE replacements.

In an embodiment, disclosed herein are methods to produce at least one scalable chemical processing technology that can depolymerize and separate building blocks from current and emerging streams of plastics with ester linkages.

In an embodiment, disclosed herein are methods to refine catalysis-enabled approaches for conversion of the intermediates into either the same recyclable plastic (closed-loop) or a polyethylene (PE)-like replacement (open-loop), with a focus on film application.

In an embodiment, the methods disclosed herein can be used to provide a rigorous analysis that quantifies the economics, energy usage, and greenhouse gas (GHG) emissions for recycling and remanufacturing of both closed-loop recycling options and open-loop upcycling options.

In an embodiment, disclosed herein is a method for feedstock-agnostic deconstruction, separations, and remanufacturing processes for polyesters.

In an embodiment, disclosed herein is a method for downstream processing (DSP) developments for recovery of individual or mixed intermediates derived from plastics with ester-linkages.

In an embodiment, disclosed herein is a method for remanufacturing of the recovered compounds into closed-loop polymers through chemical synthesis routes.

In an embodiment, disclosed herein is a method for chemical deconstruction of mixed polyesters via chemo-catalytic process development.

In an embodiment, disclosed herein is a method for synthesis and performance testing for emerging PE replacements.

In an embodiment, disclosed herein is a method for designing experimental efforts to synthesize two PE replacements.

In an embodiment, disclosed herein is a method for designing experimental efforts to understand if mixed chain-length substrates can enable PE-like materials.

In an embodiment, disclosed herein are methods for film processing and characterization of recyclable-by-design PE replacements.

In an embodiment disclosed herein are methods for making dimethyl terephthalate (DMT) from deconstruction of PET as disclosed in Scheme 1:

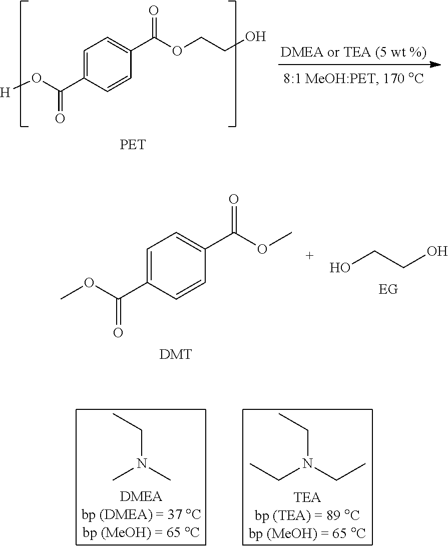

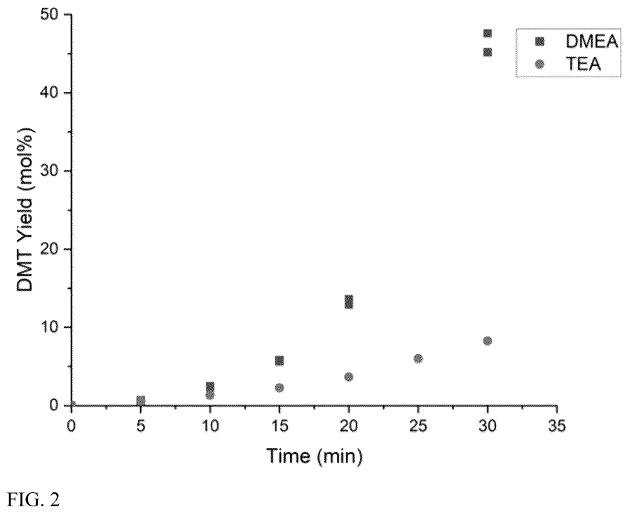

FIG. 2 depicts the DMT yield over time using the process depicted in Scheme 1.

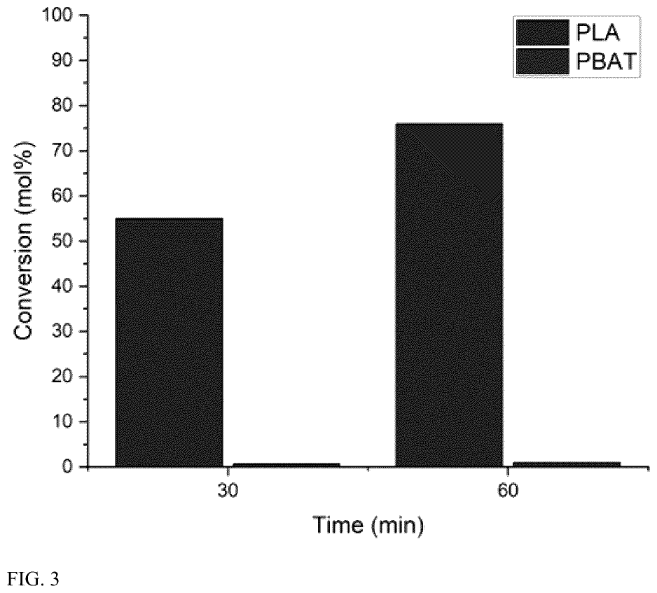

In an embodiment disclosed herein are methods for conversion of PLA and PBAT to products using methods disclosed herein, see FIG. 3 and, in an embodiment, as disclosed in Scheme 2:

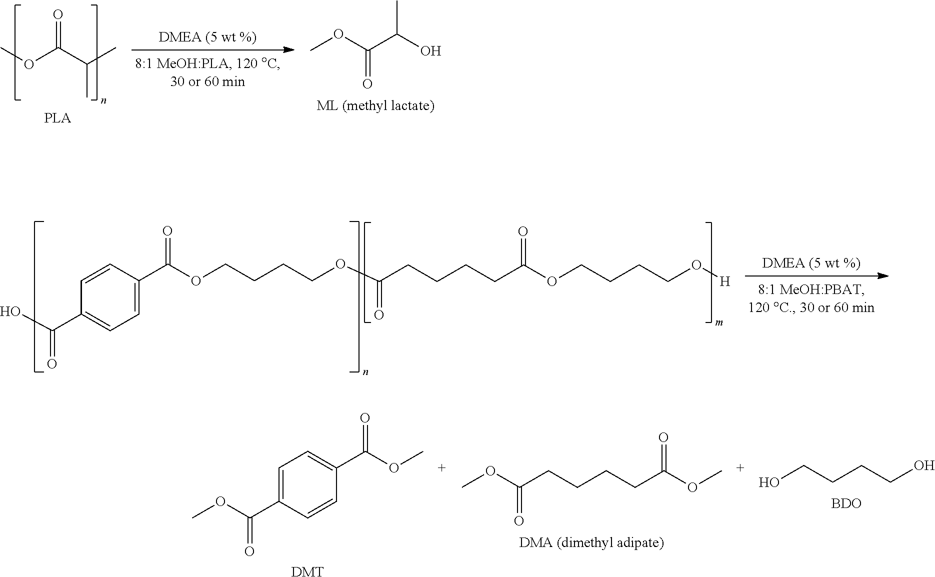

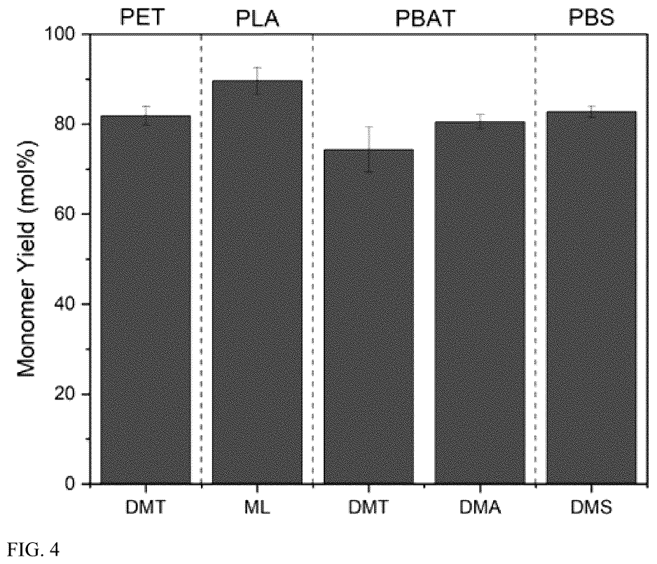

In an embodiment disclosed herein are methods for conversion of PET or PLA or PBAT or PBS to products using methods disclosed herein, see FIG. 4 and, in an embodiment, as disclosed in Scheme 3:

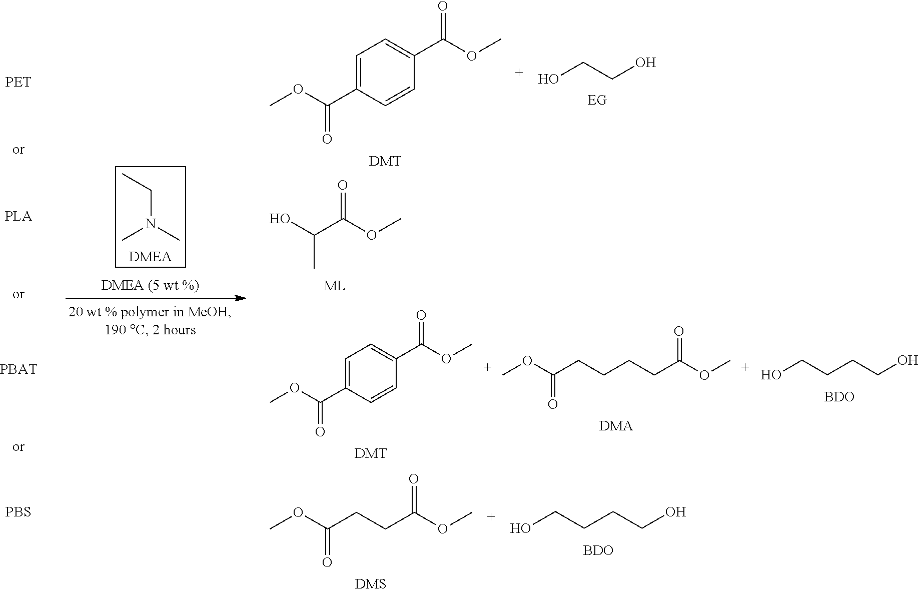

As depicted in FIG. 4, via the reaction conditions depicted in Scheme 3, PET is converted to DMT at about a 82 mole percent monomer yield; PLA is converted to ML at about a 90 mole percent yield; PBAT is converted to DMT and DMA at about a 70 mole percent yield and a 80 mole percent yield, respectively; and PBS is converted to DMS at about a 80 mole percent yield.

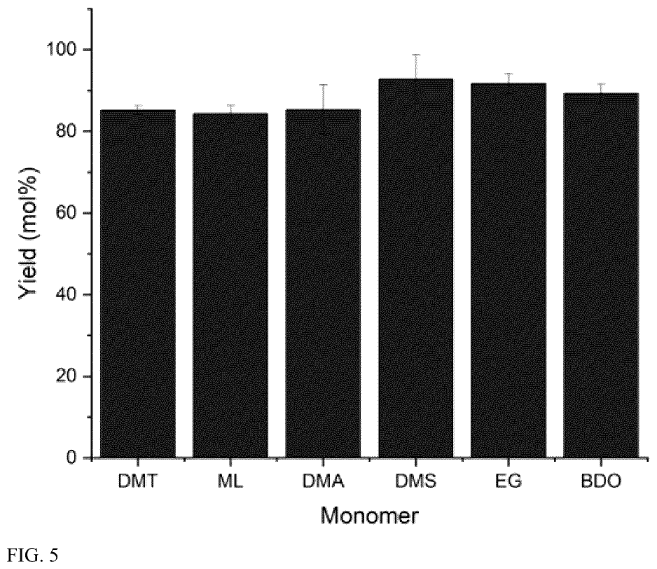

As depicted in FIG. 5, via the reaction conditions depicted in Scheme 4, a mixture of PET:PLA:PBAT:PBS at relative molar ratios of 70:14:14:2, respectively, are converted to DMT, ML, DMA, DMS, EG and BDO monomers using DMEA at 5 weight percent; and 25 weight percent polymer in MeOH at 170 degrees Celsius for 3 hours. The mole percent yield of the monomer products from the conversion of PET:PLA:PBAT:PBS at relative molar ratios of 70:14:14:2, respectively, under the reaction conditions of Scheme 4 are about 84 mole percent DMT; 82 mole percent ML; 84 mole percent DMA; 92 mole percent DMS; 90 mole percent EG; and 88 mole percent BDO.

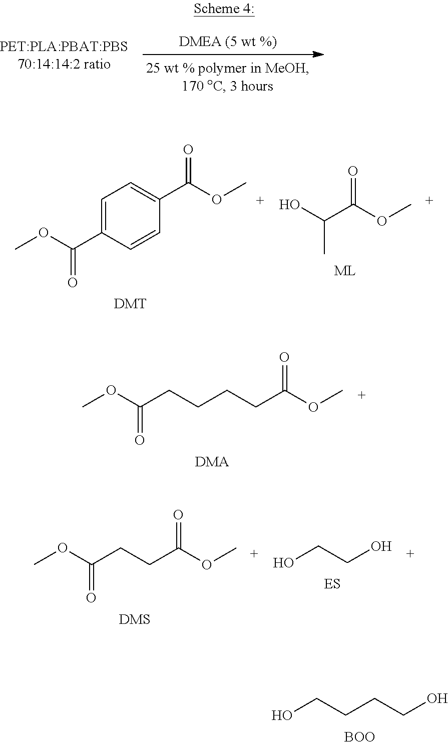

Accordingly, in an embodiment, using methods disclosed herein polymers containing ester bonds can be depolymerized in separate reactions for each polymer (see Scheme 3, for example) or may be depolymerized in one reaction containing a mixture of multiple polymers (see Scheme 4, for example).

In an embodiment, polymers may be deconstructed to their monomeric components by using any of the Schemes disclosed herein. Any polymer containing an ester bond may be deconstructed using the reaction schemes and conditions disclosed herein. Non-limiting examples of polymers that may be deconstructed into their monomers include PET, PVL, PBT, PET, PLA, PBAT and PBS.

In an embodiment, amines useful in the deconstruction schemes and methods disclosed herein have boiling points that enable separation from the substrates, products and solutions in the various schemes disclosed herein via distillation. In an embodiment the amines used in the schemes and methods disclosed herein are tertiary amines. In another embodiment, the amines used in the methods and schemes disclosed herein are DMEA or TEA.

The foregoing disclosure has been set forth merely to illustrate the invention and is not intended to be limiting.

We claim:

1. A method for converting a polymer containing an ester-bond into monomers comprising the step of contacting the polymer with an amine in a solution comprising alcohol at a temperature of from 100 to 190 degrees Celsius.

2. The method of claim 1 wherein the alcohol is methanol.

3. The method of claim 1 wherein the amine is a tertiary amine.

4. The method of claim 1 wherein the amine is selected from the group consisting of DMEA or TEA.

5. The method of claim 1 wherein the boiling point of the amine differs from the boiling point of the alcohol by at least 24 degrees Celsius.

6. The method of claim 1 wherein the polymer is selected from the group consisting of PET, PVL, PBT, PET, PLA, PBAT, PHAs, PCL and PBS.

7. The method of claim 1 wherein the monomers are selected from the group consisting of DMT, ML, DMA, DMS, EG and BDO.

8. The method of claim 1 wherein the molar ratio of alcohol to the polymer is 8 to 1.

9. The method of claim 1 wherein the weight percent of the amine is about 5 weight percent of the weight of the reaction components.

10. The method of claim 1 wherein the weight percent of the polymer comprises 20 to 60 weight percent of the reaction components.

11. The method of claim 1 wherein about 55 mole percent of the polymer is converted into monomers after a reaction time of about 30 minutes.

12. The method of claim 1 wherein about 70 mole percent of the polymer is converted into monomers after a reaction time of about 2 hours.

13. A method for converting a mixture of different ester-bond containing polymers into monomers comprising the step of contacting the mixture of polymers with an amine in a solution comprising alcohol at a temperature of from 100 to 190 degrees Celsius.

14. The method of claim 13 wherein the alcohol is methanol.

15. The method of claim 13 wherein the amine is a tertiary amine.

16. The method of claim 13 wherein the amine is selected from the group consisting of DMEA or TEA.

17. The method of claim 13 wherein the boiling point of the amine differs from the boiling point of the alcohol by at least 24 degrees Celsius.

18. The method of claim 13 wherein the mixture of polymers contains polymers selected from the group consisting of PET, PVL, PBT, PET, PLA, PBAT, PHAs, PCL and PBS.

19. The method of claim 13 wherein the weight percent of the amine is about 5 weight percent of the reaction components; and wherein the mixture of polymers comprises 20 to 60 weight percent of the weight of the reaction components.

20. The method of claim 13 wherein greater than about 80 mole percent of the polymer mixture is converted into monomers after about 3 hours of contacting the mixture of polymers with an amine in a solution comprising alcohol at a temperature of from 100 to 190 degrees Celsius.
